(19) United States
(12) Patent Application Publication
(10) Pub. No.: US 2001/0051800 A1
(43) Pub. Date: Dec. 13, 2001

(54) Method for joining biological tissues

(75) Inventors: Karpov Eugeny, Moscow (RU); Reztsov Alexander, Moscow (RU)

Correspondence Address: ROBERT W. BECKER & ASSOCIATES, 11896 N. Highway 14, Suite B, Tijeras, NM, 87059, US

(73) Assignee: Firma Biomedy AG, Zug (CH)

(21) Appl. No.: 09/813,038

(22) Filed: Mar. 19, 2001

(30) Foreign Application Priority Data
Jun. 13, 2000 (RU) 2000-114 847

(51) Int. Cl. A61B 18/20

(52) U.S. Cl. 606/8

(57) ABSTRACT

A method is provided for obtaining a quality discontinuous surgical suture of biological tissues. The method includes treating the biological tissues with a laser beam having a power flux density of 1-7 kW/cm², preferably 3-5 kW/cm², on the surface of the joined biological tissues. To form a discontinuous surgical suture, a laser is used which is powered by copper vapors for generating a laser beam at a wavelength of 0.5-0.6 microns in combination with short-focus optics. The source of laser radiation has a radiation power of 5-15 W, preferably 8-11 W, at a periodic pulse operating mode of laser beam generation and at a focal distance of the focusing objective of 3-7 cm and the exposure period of the biological tissues being treated by the laser radiation is 3-15 seconds.

### BACKGROUND OF THE INVENTION

The present invention relates to a method having application in the field of medicine and, more particularly, in the field of surgery involving a laser radiation source operable to form a surgical suture.

White R. A. et, Mechanism Of Tissue Fusion In Argon Laser-Welded Vein-Artery Anastomoses, Lasers Surg. Med., 1988, v. 8, pages 83-89, describes a method of joining biological tissues by “welding” thereof with the aid of a source of laser radiation. However, this method results in a condition at the “welded” joined tissues in which the supply of blood thereto is not satisfactory.

Russian Federation Application No. 96124526/14, publ. BI No.

6, 1999, describes a method of joining biological tissues by treatment of the biological tissues with a laser beam so as to form a discontinuous surgical suture. Biological tissues joined by this method benefit from the preservation of blood flow thereto which has a beneficial effect on the process of accretion of the joined biological tissues. However, the noted Russian Federation Application No. 96124526/14 does not disclose the operating parameters of the process for forming the discontinuous surgical suture by means of the source of laser radiation.

### SUMMARY OF THE INVENTION

An object of the method of the present invention is to provide a suitable set of operational parameters for obtaining a quality discontinuous surgical suture by performance of the method.

In accordance with one aspect of the method of the present invention, the method can be performed to join biological tissues with a plurality of discontinuous surgical sutures by treatment of the biological tissues with a laser beam having a power flux density of 1-7 kW/cm², preferably 3-5 kW/cm², on the surface of the joined biological tissues.

In accordance with another aspect of the method of the present invention, the source of laser radiation is a laser powered by copper vapors for generating a laser beam at a wavelength of 0.5-0.6 microns in combination with short-focus optics.

In accordance with a further aspect of the present invention, the source of laser radiation has a radiation power of 5-15 W, preferably 811 W, at a periodic pulse operating mode of laser beam generation and at a focal distance of the focusing objective of 3-7 cm and that the exposure period of the biological tissues being treated by the laser radiation is 3-15 seconds.

### BRIEF DESCRIPTION OF THE DRAWINGS

FIG. 1 is a schematic view of a joint structure formed by laser beam treatment of biological tissues for interconnecting the biological tissues, the joint having the configuration of a “hollow rivet”;

FIG. 2 is a plot of estimates of the power flux density for an unfocused laser beam;

FIG. 3 is a plot of estimates of the power flux density of a focused laser beam; and

FIG. 4 is a series of illustrations of the progressive configuration of a welded suture as its strength is tested with the aid of air overpressure.

### DETAILED DESCRIPTION OF THE PREFERRED EMBODIMENT

Experimental studies were conducted in the Laboratory of Cellular and Molecular Pathology of the Scientific Center of the Moscow Medical Academy Setchenov.

The following lasers were used for the laser “welding” of biological tissues in a medical field context:

gas CO₂-lasers (□=10.6 microns),

neodymium lasers (□=2.94 microns, 2.06 microns, 1.32 microns, 1.06 microns, 0.53 microns),

semiconductor lasers (□=0.81 microns),

argon lasers (□=0.51 microns, 0.49 microns)

A measure of the usefulness of a selected laser for the joining of biological tissues is the efficiency of the delivery of the requisite energy into the treated area for effecting the joining of the biological tissues. On the one hand, the wavelength of the laser radiation should provide uniform energy distribution on the entirety of the treated area while, on the other hand, the amount of energy absorbed by the biological tissues should be sufficient to effect the joining of the biological tissues. The achievement of these two objectives is dependent in many respects upon the power (energy) of the laser radiation delivered to the treated area, as well as upon the space and time distribution of the energy in the treated area.

One conclusion drawn from the thus performed studies is that it is most advantageous to use a laser beam produced with the aid of copper vapors to form a discontinuous surgical suture. It should be mentioned in this context that a laser of this type had not been earlier applied in a medical context.

The results of three experimental protocols which explored operational parameters of the method of the present invention are set forth below.

Experimental Protocol 1

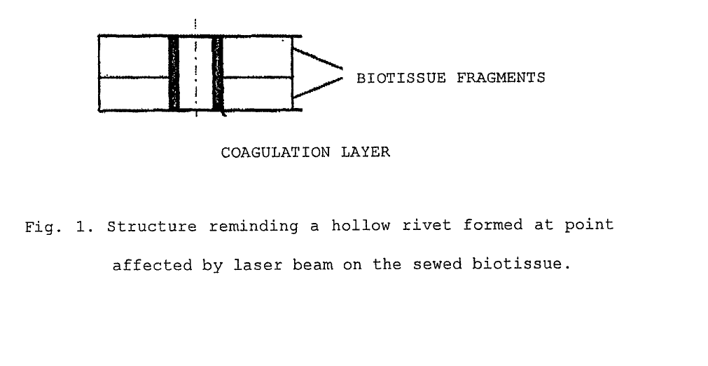

To explore one aspect of the operational parameters of the method of the present invention, the biological tissues of internal organs of laboratory animals (rats) were treated with laser radiation to effect the formation of interconnecting joints between the treated biological tissues which have the configuration of a “hollow rivet” (see FIG. 1).

The requisite strength and density of the laser formed suture were ensured by the selection of an appropriate frequency of the “hollow rivet” joints and appropriate sizing of the rings thereadjacent formed of denatured albumen.

A relatively thin stratum of necrotized tissue formed as a result of heating ensures the necessary mechanical strength of the suture at the initial stage of healing. After the walls of the “rivet” are destroyed, the strength of the tissue joint is dependent upon the size of the zone of structural reorganization of the albumen and the extent of the denaturation thereof.

Spectral and polarizing studies of the applicants have shown that biological tissues have relative transmittance. Thus, laser radiation of the visible and the near IR wavelength penetrates relatively deeply into the biological tissues. In view of the fact that the laser radiation propagates into the biological tissues at a speed close to the speed of light, the laser beam and, consequently, the optical power, is delivered into the biological tissues practically instantaneously. Accordingly, the heating of the biological tissues commences simultaneously throughout the entirety of the tissue lying within the borders of the localization of the energy of the laser radiation.

A vaporization of the biological tissues occurs in the central zone of the laser beam, at high power flux density, and a melting of the biological tissues occurs on the periphery of the beam. Studies of the applicants have established that the ratio between the thickness of the joined biological tissues and the cross sectional size of the laser beam should be (5-7): (0.8-1.2). Accordingly, it is believed that the distribution of the power flux density of the laser beam in its longitudinal direction determines the uniformity of the energy conversion efficiency in the joining zone.

A uniform distribution of the energy conversion efficiency along the beam should result in only minimal differences in the mechanical stresses in the joined biological tissues. Thus, it is believed that such a condition best ensures strong welding or joining of the biological tissues. Apparently, a desirably strong welded or joined condition can be effected so long as the power flux density varies only weakly in the biological tissues within the path of the laser beam.

From the perspective of achieving a laser joining of biological tissues, the choice of the preferred wavelength of the laser radiation was made by taking into account the shape of the laser beam penetrating the biological tissues. In this regard, it was assumed that the variation of power of the laser radiation at its propagation in the biological tissues conforms to Bouguer's law.

The distribution of the power flux density of the laser radiation on the biological tissues was studied at different wavelengths of unfocused and focused laser beams and took into account the data obtained from the experimental study of the optical characteristics of the biological tissues. In connection with the study of focused laser beams, it was specified that the diameter of the beam at the input into the focusing optics was 2 cm, that the focal distance was 5 cm, and that the cross sectional extent of the beam in the zone of the focal beam constriction was 0.03 cm.

The power flux density in any given section of the biological tissue P(x) was normalized relative to the power flux density on the surface of the biological tissue Po for the wavelengths of 0.5 microns, 0.6 microns, 0.7 microns, and 0.9 microns.

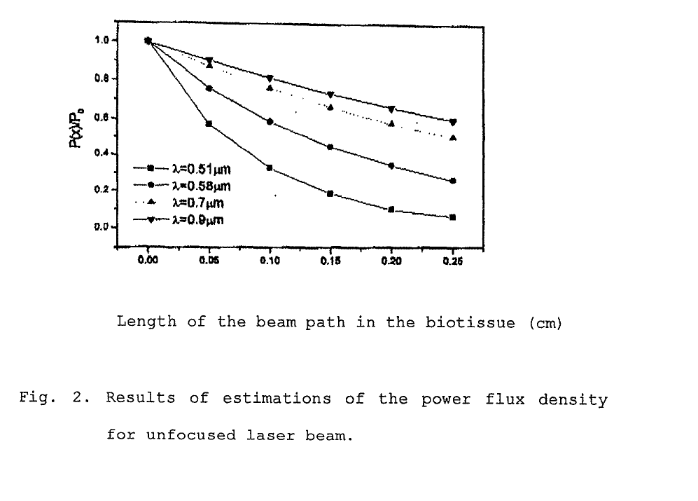

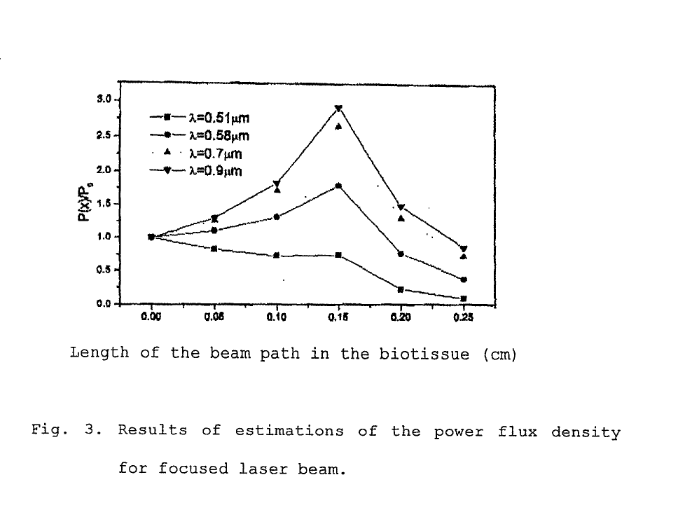

The results of the study of unfocused laser beams and focused laser beams are shown in FIGS. 2 and 3, respectively.

As seen in the plot of the results concerning the unfocused laser beam studies shown in FIG. 2, a laser beam radiation having a wavelength of 0.9 microns is to be preferred. The gradient of the power flux density along the laser beam increases in correspondence with the decrease of the wavelength of the laser beam. The largest gradient was observed at a laser beam wavelength of 0.5 microns. The advantage that apparently obtains in radiating in the near IR wavelength band is that it avoids an essential problem relating to long focus laser beam operation. In long focus laser beam operation, a space distribution can be formed. In this mode of operation, the dimension of the beam in a zone of focal beam constriction is relatively larger than that of the beam in a short focus mode of operation. Therefore, it is necessary to increase the exposure time or the power of the laser radiation in order to provide optimal energy-conversion efficiency. However, increasing the exposure time decreases the operational speed of the laser “welding” or joining and increasing the power of the laser radiation complicates the operating conditions of the laser delivery device used for the joining of biological tissues.

In a short focus laser beam operation with a focused beam, a more uniform distribution of the power flux density can be provided at the wavelength of 0.5 microns, as can be seen in FIG. 3. The gradient of the power flux density along the laser beam increases in correspondence with increasing wavelength. It appears that radiation in the visible wavelength bandwidth of 0.5-0.6 microns is to be preferred over radiation in the near IR wavelength bandwidth. Moreover, the advantages of this preferential wavelength bandwidth become even more apparent when considered in light of the possibility of providing high power densities at small power of the laser in short focus laser beam operations.

Experimental Protocol 2

Experimental studies of different operational modes of focusing laser radiation were carried out to determine the geometrical characteristics of a laser beam which could perform a biological tissue joining operation. The possibility of biological tissue joining by a laser beam was explored in both the long focus operational mode and the short focus operational mode.

Experiments were carried out in the long focus operational mode at a laser radiation power of 4 W and a focal distance of the focusing objective of 80 cm. The exposure time varied from 20 seconds to 130 seconds. The power flux density on the surface of the biological tissue was 625 W/cm².

A burning through of the biological tissues was observed in all cases. Welding or joining of the biological tissues was not observed. The area of interaction of the laser beam with the biological tissue was characterized by a clearly defined through hole and a peripheral zone. The internal wall and the peripheral zone were carbonized.

Experiments were carried out in the short focus operational mode at a laser radiation power of 10.8 W and a focal distance of the focusing objective of 5 cm. The exposure time varied from 1.5 seconds to 30 seconds. The power flux density on the surface of the biological tissue was 1-7 kkW/cm². Samples were studied at the following exposures: 2 samples—1.5 seconds; 7 samples—5 seconds; 1 sample—10 seconds; 5 samples—20 seconds; and 1 sample—30 seconds.

Burning through of the biological tissues and the welding or joining together thereof was observed in all cases. The diameter of the biological tissue burn through increased in correspondence with the increase in the exposure length. In contrast to that observed in connection with the exposure time of 20 seconds, no carbonization of the biological tissue was observed at the exposure times of 1.5 seconds and 5 seconds.

The following observations can be drawn in connection with the experimental results of the studies of the long focus and short focus laser beam operational modes. Although the energy conversion efficiency in the long focus operational mode at the exposure time of 20 seconds approached that of the short focus operational mode at the exposure time of 5 seconds (12.5 kJ/cm²), “welding” or joining of the biological tissues was observed only in the case of the short focus operational mode. It appears that space distribution of the power flux density of the laser beam radiation along its direction of propagation is important for the “welding” or joining of biological tissues.

In subsequent experiments, the exposure time of the biological tissues treated by laser radiation varied from 5 seconds to 15 seconds at a laser power radiation of 5-15 W. The purpose of these experiments was to determine the range of exposure times which yield a satisfactory welding or joining of the biological tissues.

These subsequent experiments were carried out on 9 groups of biological tissues differing from one another essentially in morphological characteristics such as color, pattern of fibers, and the presence of fatty deposits.

Steady welding or joining of the biological tissues was observed in all cases in the range of exposure times from 5 seconds to 15 seconds.

Experimental Protocol 3

The quality of the laser welding was estimated based upon the results of strength testing of the weld sutures and analysis of the biological state of the zone of the suture.

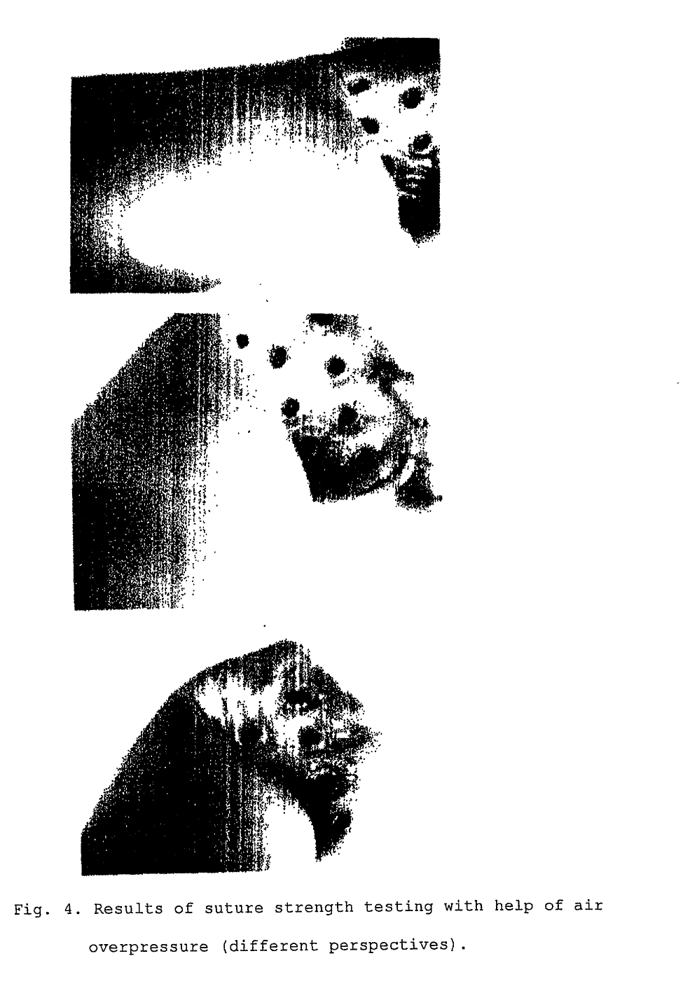

The strength of the weld joints was tested by producing an overpressure of up to 0.1 bar in the zone of the suture. In this regard, the tested sutures were comprised on the biological tissues welded at laser power radiations of 5-15 W and at exposure times of 5-15 seconds. FIG. 4 shows the images of a biological tissue in the course of strength testing thereof. In all cases, the suture maintained its mechanical characteristics and was not destroyed.

The strength tests thus performed confirmed the strength of the weld joints in the range of the applied overpressures.

The exposure times were varied from 1 second to 32 seconds in connection with the study of the biological state of the welding zones. The laser radiation power was 5-15 W.

Portions of internal organs of rats which had previously been placed in physiological solution were used to define the biological state of the biological tissue after the biological tissue had been treated by the laser beam radiation so as to form weld sutures configured as “hollow rivets”.

The laser radiation power and the exposure time were varied in the experiments. At the laser radiation power of 8-11 W, the exposure time was at a value of 1-15 seconds, and at the laser radiation power of 4-6 W, the exposure time was at a value of 8-32 seconds. Disruption of the biological tissue was observed at the laser radiation power of 8-11 W during the exposure time values of 5-10 seconds. Disruption occurred much later at the lower laser radiation power values during the exposure time values of 30-32 seconds.

The experimental studies suggest that, to obtain a quality discontinuous surgical suture by performance of the method of the present invention, it is necessary to treat the biological tissues with a laser beam having a power flux density of 1-7 kW/cm², preferably 3-5 kW/cm², on the surface of the joined biological tissues. To form a discontinuous surgical suture, it is recommended that a laser is used which is powered by copper vapors for generating a laser beam at a wavelength of 0.5-0.6 microns in combination with short-focus optics. It is recommended that the source of laser radiation has a radiation power of 5-15 W, preferably 8-11 W, at a periodic pulse operating mode of laser beam generation and at a focal distance of the focusing objective of 3-7 cm and that the exposure period of the biological tissues being treated by the laser radiation is 3-15 seconds.

Thus, the method of the present invention permits one to obtain a quality discontinuous surgical suture with the use of laser radiation.

The specification incorporates by reference the disclosure of Russian priority document 2000-114-847 of Jun. 13, 2000.

The present invention is, of course, in no way restricted to the specific disclosure of the specification and drawings, but also encompasses any modifications within the scope of the appended claims.

What we claim is:

1. A method of joining biological tissues comprising:
treating the biological tissues to be joined with a laser beam having a power flux density of 1-7 kW/cm² on the surface of the joined biological tissues.

2. A method according to claim 1 wherein the step of treating the biological tissues includes treating the biological tissues to be joined with a laser beam having a power flux density of 3-5 kW/cm² on the surface of the joined biological tissues.

3. A method according to claim 1, wherein the step of treating the biological tissues includes treating the biological tissues with a laser which is powered by copper vapors and generates a laser beam at a wavelength of 0.5-0.6 microns in combination with short-focus optics.

4. A method according to claim 1, wherein the step of treating the biological tissues includes treating the biological tissues with a laser having a laser radiation power of 5-15 W at a periodic pulse operating mode of laser beam generation.

5. A method according to claim 1, wherein the step of treating the biological tissues includes treating the biological tissues with a laser having a laser radiation power of 8-11 W at a periodic pulse operating mode of laser beam generation.

6. A method according to claim 1, wherein the step of treating the biological tissues includes treating the biological tissues with a laser beam generated at a focal distance of the focusing objective of 3-7 cm.

7. A method according to claim 1, wherein the step of treating the biological tissues includes treating the biological tissues with a laser beam for an exposure period of 3-15 seconds.
